(19) United States
(12) Patent Application Publication
(10) Pub. No.: US 2007/0017039 A1
(43) Pub. Date: Jan. 25, 2007

(54) Methods of merchandising hair color refresher products

(76) Inventors: Pauline Jane Errey, New Canaan, CT (US); Monica Agrawal, Stamford, CT (US); Mona Klein Klapper, Rye Brook, NY (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/187,554

(22) Filed: Jul. 22, 2005

(51) Int. Cl. A61K 8/00

(52) U.S. Cl. 8/405

(57) ABSTRACT

A method of merchandising a hair color refresher product to a consumer having oxidatively colored hair of an initial color and/or an initial shine imparted by a previous oxidative hair coloring treatment comprises the steps of: (a) providing the hair color refresher product in kit form at a purchase location at which oxidative hair coloring products in kit form also are provided; (b) communicating to the consumer that the hair color refresher product is used on the hair, at a time after the previous oxidative hair coloring treatment but before a subsequent oxidative hair coloring treatment, to refresh the initial color of the hair of the consumer; and, optionally, (c) providing one or more indicia to facilitate selection by the consumer of a suitable shade of the hair color refresher product.

### FIELD OF THE INVENTION

The present invention relates to hair color refresher products and methods of merchandising such products.

### BACKGROUND OF THE INVENTION

The alteration of the color of keratinous fibers, in particular human hair, by the application of hair dyes is well known. In order to provide the consumer with the hair color and the intensity of color desired, a very complex chemical process is utilized. Non-permanent hair colorant formulations typically comprise non-permanent dyes such as direct dyes or pigments which are deposited on to the surface of the hair and are gradually washed out over successive washing cycles. Permanent hair colorant formulations typically comprise oxidative hair dye precursors, which can diffuse into the hair through the cuticle and into the cortex where they can then react with each other and suitable oxidizing agents to form the end dye molecules. Due to the large size of these resultant molecules they are unable to readily diffuse out of the hair during subsequent washing with water and/or detergents; hence delivering the consumer-desired permanency of color. The reaction typically takes place in an aggressive environment at approximately pH 8 to 11 in the presence of an alkalizing agent and in the presence of an oxidizing agent. Typically, the consumer repeats this process, for example, every six to eight weeks, in order to maintain the desired hair color shade and the intensity of color and to ensure continual, even coverage of the hair including coverage of new hair growth.

Despite the fact that hair colorant products have been available for many years, the products still exhibit a number of consumer-related deficiencies. For example, the initial color result, obtained immediately after coloring the hair, changes over time during the subsequent days and weeks resulting in both a change in color intensity and shine, and color tonality of the hair.

Many attempts have been made to improve the color profile provided by hair colorant products. These have typically resided in the development of new dye precursors, and combinations of dye precursors to enhance the so called “wash-fade” profile. Despite offering some advantages, this approach does not provide sufficient advantages in color maintenance over time, and is not effective for all desired shades. Alternatively, the use of oxidative dyes in combination with pre-formed direct dyes within a single composition or as separate application steps has been taught. Such products may yield more intense initial color, but still do not provide the long term desired color profile. Such products also may result in unacceptable variations in root to tip color uptake.

Attempts also have been made to “seal” in the color delivered by hair colorant products with conditioning treatments, which improve the hair condition. While this provides some limited improvement of color maintenance over time, it is not sufficient to mitigate all of the current deficiencies observed by the consumer.

An alternative approach to address the above problems associated with a lack of maintenance of the initial color result on colored hair, however, is to “refresh” the color and the shine, which is obtained immediately after the initial coloring of the hair, with a refresher treatment.

Professional hair salons offer a treatment known as “glazing”, which can be used to refresh the color or shine of a consumer's hair. Typically, a professional colorist mixes a semi- or demi-permanent hair dye composition in a shampoo base and applies it to a consumer's hair. For such a glazing treatment, the consumer relies upon the professional colorist to customize a hair dye composition which is appropriate to provide the consumer's desired refreshed color results. Although glazing treatments can provide a satisfactorily refreshed look to the color or shine of a consumer's hair, such treatments currently are only available from a professional hair salon. This is disadvantageous for many consumers, as visiting a salon to receive a glazing treatment is time-consuming and expensive.

At-home retail hair coloring products, commonly provided in a kit, are known and have been offered for many years as an alternative to professional salon hair coloring treatments. Generally, though, all of such retail hair coloring products, whether imparting semi-permanent, demi-permanent, or permanent color to a consumer's hair, are marketed to prospective consumers as hair coloring treatments providing an initial color result. No current retail hair coloring products are marketed to prospective consumers as providing a treatment to refresh the color or the shine of the initial color result which has changed over time subsequent to the initial hair coloring treatment. In other words, no at-home hair color treatment product or kit which provides results analogous to a professional salon glazing treatment is currently marketed.

Recently, an at-home retail hair coloring product, known as L'Oreal Paris® Tone Refiner™ balancing toning gloss, has been introduced. This product claims to tone down dullness, brassiness, and unwanted “warmth” of already colored or highlighted hair. While this product is not marketed as providing an initial color result, it does not claim to provide an increase in hair color along with an increase in hair shine to remedy the fading of the hair color and the hair shine over time. Rather, in toning down brassiness and warmth, it actually results in a perceived decrease in hair color. Further, this product is marketed as being suitable for use on the same day as coloring, lightening, or highlighting. Thus, it is not positioned to address the problem of the fading of hair color and hair shine during the days and weeks subsequent to an oxidative hair coloring treatment.

Consequently, a need exists for an at-home retail hair color refresher product which provides a treatment to refresh the color or the shine of an initial color result imparted by a previous hair coloring treatment. Advantageously, such a retail hair color refresher product should be easy and quick to use and relatively inexpensive.

As such a retail hair color refresher product is not currently marketed, however, it is recognized that many prospective consumers may not immediately appreciate how this new-to-the-retail-market product is different from currently marketed products. Further, many prospective consumers may not immediately appreciate how this new-to-the-retail-market product works to remedy an inadequacy of some of those currently marketed products, and how its use complements the use of some of those currently marketed products. Thus, a need exists to communicate to prospective consumers the benefits of using the retail hair color refresher product. There also is a need to communicate to prospective consumers when the retail hair color refresher product should be used to provide such benefits.

While such a need exists for a retail hair color refresher product, it also is recognized that different consumers have different initial hair colors, each of which may require a different treatment to refresh the color or the shine of that respective initial hair color. Thus, a need also exists for a retail hair color refresher product which is provided in a plurality of shades, each of the plurality of shades being suitable for refreshing the color or the shine of at least one of the possible different initial hair colors of consumers.

It further is recognized, however, that a consumer who receives a glazing treatment at a professional hair salon benefits from the professional colorist selecting and preparing a specific treatment composition suitable for that consumer's desired results. In contrast, a prospective consumer of a retail hair color refresher product, which is provided for sale in, for example, a pharmacy, a department store, or another typical retail outlet, likely would have to select a suitable shade without assistance from a professional colorist. As a result, a need exists for means to facilitate selection by a prospective consumer of a shade suitable to provide that consumer's desired results.

Accordingly, a need exists for a retail hair color refresher product which refreshes the color or the shine of an initial color imparted to hair by a previous hair coloring treatment. In turn, there exists a need for merchandising such a hair color refresher product according to a method which provides for one or more of the above requirements.

### SUMMARY OF THE INVENTION

The present invention relates to methods of merchandising a hair color refresher product to a prospective consumer having oxidatively colored hair of an initial color and/or an initial shine imparted by a previous oxidative hair coloring treatment. In one embodiment of the present invention, the method comprises the steps of:

(a) providing the hair color refresher product in kit form at a purchase location at which a plurality of oxidative hair coloring products in kit form also are provided, the hair color refresher product comprising:
(i) an individually packaged hair color refresher component comprising a hair color refresher composition, the hair color refresher composition comprising at least one preformed or water soluble or water dispersible dye, and
(ii) instructions for use which direct a user of the hair color refresher product to apply the hair color refresher composition to the hair of the user and to retain the hair color refresher composition on the hair of the user for a period of time of about 15 minutes or less, and then to rinse the hair color refresher composition from the hair of the user, and wherein the hair color refresher product is provided in a plurality of general shades, at least one of the plurality of general shades being suitable for refreshing the initial color of the hair of the prospective consumer;

(b) communicating to the prospective consumer that the hair color refresher product is used on the hair of the prospective consumer, at a time after the previous oxidative hair coloring treatment but before a subsequent oxidative hair coloring treatment, to refresh the initial color of the hair of the prospective consumer; and

(c) providing one or more indicia in association with each of the plurality of general shades of the color refresher product to facilitate selection by the prospective consumer of the one of the plurality of general shades of the hair color refresher product which is suitable for refreshing the initial color of the hair of the prospective consumer.

In another embodiment of the present invention, the method of merchandising comprises the steps of:

(a) providing the hair color refresher product in kit form at a retail purchase location at which a plurality of oxidative hair coloring products in kit form also are provided, the hair color refresher product comprising:
(i) an individually packaged hair color refresher component comprising a hair color refresher composition, the hair color refresher composition comprising at least one preformed or water soluble or water dispersible dye, and
(ii) instructions for use which direct a user of the hair color refresher product to apply the hair color refresher composition to the hair of the user and to retain the hair color refresher composition on the hair of the user for a period of time of about 15 minutes or less, and then to rinse the hair color refresher composition from the hair of said user; and

(b) communicating to the prospective consumer that the hair color refresher product is used on the hair of the prospective consumer, at a time after the previous oxidative hair coloring treatment but before a subsequent oxidative hair coloring treatment, to refresh the initial color of the hair of the prospective consumer.

It is believed that the methods of the present invention create improved opportunity to generate profit through increased sales of products relating to consumer hair coloring. As a result of the steps of the method of the present invention, a new-to-retail-market hair color refresher product is provided for sale advantageously at a purchase location at which known retail hair coloring products also are provided for sale. Additionally, a prospective consumer is educated about how the new hair color refresher product can address a currently unmet consumer need, is encouraged to purchase and to try the new hair color refresher product, and, optionally, is assisted in selecting a version of the new hair color refresher product suitable to provide the results desired by the consumer.

### DETAILED DESCRIPTION OF THE INVENTION

While the specification concludes with claims that particularly point out and distinctly claim the invention, it is believed the present invention will be better understood from the following description.

As used herein, the term “hair” refers to keratinous fibers on a living body, e.g., a person, or a non-living body, e.g., in a wig, hairpiece, or other aggregation of non-living keratinous fibers. Mammalian, preferably human, hair is preferred. Notably, hair, wool, fur, and other keratinous fibers are suitable substrates for treatment by the hair color refresher products described herein.

As used herein, the term “merchandising” refers to the act of selling goods for profit in combination with the act of promoting the sale of those goods, such as by advertising.

As used herein, the term “prospective consumer” refers to any living person who has an actual or potential capacity to purchase the hair color refresher product of the present invention.

As used herein, the term “oxidative hair coloring product” refers to any retail product or kit which can be used by a consumer to perform a permanent (i.e., Level 3) or a demi-permanent (i.e., Level 2) at-home hair coloring treatment.

As used herein, the term “oxidative hair coloring treatment” refers to an act of coloring the hair with a mixture of at least one oxidizing agent and at least one oxidative dye, and, optionally, at least one alkalizing agent. This term includes permanent (i.e., Level 3) and demi-permanent (i.e., Level 2) color treatments. Unless otherwise specifically limited, this term applies equally to treatments performed by a professional colorist in a salon as well as to treatments performed at home with the use of a typical retail hair coloring kit.

As used herein, the term “previous oxidative hair coloring treatment” refers to an oxidative hair coloring treatment performed on a consumer's hair at a time more than about 24 hours prior to the time of use of the hair color refresher product of the present invention.

As used herein, the term “immediately after coloring” or “immediately after a hair coloring treatment” refers to a point in time within the range of about 1 second to about 24 hours subsequent to a respective hair coloring treatment.

As used herein, the term “consumer-observable” or “consumer-observably” refers to an observation within the ordinary perception of an average consumer.

As used herein, the terms “initial color” and “initial shine” refer to the consumer-observable color and the consumer-observable shine, respectively, of a consumer's hair as perceived immediately after a respective oxidative hair coloring treatment.

As used herein, the term “fade” or “fading” refers to a consumer-observable decrease in the color or the shine of previously oxidatively colored hair, relative to the initial color or the initial shine observed immediately after the previous hair coloring treatment.

As used herein, the term “refresh” refers to a consumer-observably increase or enhancement of the color or the shine of a consumer's hair, which is intended to remedy any consumer-observable fading of the initial color or the initial shine observed immediately after a previous hair coloring treatment. Preferably, the consumer-observably increase or enhancement of the color or the shine of the hair lasts for about 6 to 8 shampoos.

As used herein, the term “suitable for refreshing” a certain initial color of hair of a consumer refers to being of a certain shade formulation such that the consumer-observable increase or enhancement of the color or the shine of the hair consumer-observably matches or complements the initial color of hair.

As used herein, the term “purchase location” refers to a place at which or through which a product is made available for sale to a consumer. This term preferably includes typical retail outlets or retail stores, such as pharmacies, department stores, grocery stores, and the like. This term also includes Internet websites. Preferably, this term does not include professional hair salons.

As used herein, the term “indicia” refers to any identifying mark which is useful to a prospective consumer in distinguishing one of the plurality of general shades of the hair color refresher product from among the others, and, further which signals to the consumer that at least one of the plurality of general shades of the hair color refresher product is suitable for refreshing the initial color or the initial shine of the hair of the consumer.

All percentages, parts and ratios are based upon the total weight of the compositions of the present invention, unless otherwise specified. All such weights, as they pertain to listed ingredients, are based on the active level, and, therefore, do not include solvents or by-products that may be included in commercially available materials, unless otherwise specified. The term “weight percent” may be denoted as “wt. %” herein.

Except as otherwise noted, all amounts including parts, percentages, and proportions are understood to be modified by the word “about”, and amounts are not intended to indicate significant digits. All amounts are based on the final composition applied to the hair unless otherwise stated.

The methods of merchandising of the present invention comprise the general steps of providing the hair color refresher product at a retail purchase location, communicating to the prospective consumer when and why the hair color refresher product is used, and, optionally, providing one or more indicia to facilitate selection by the prospective consumer of a suitable shade of the hair color refresher product. Each of these steps, as well as preferred or additional elements, is described in detail hereinafter.

A. Providing the Hair Color Refresher Product

The methods of the present invention comprise the step of providing a hair color refresher product in kit form at a purchase location at which a plurality of oxidative hair coloring products in kit form also are provided. It is believed that no currently marketed at-home hair color treatment product or kit is marketed as giving results analogous to a professional salon glazing treatment. Providing such a new-to-retail-market hair color refresher product for sale at the same purchase location as known hair coloring products, to which the new product is complementary, improves opportunity to generate profit through the sales of the new hair color refresher product.

According to the present invention, the hair color refresher product is a product which is used on previously oxidatively colored hair to refresh the color or the shine of the hair in between regular or periodic oxidative hair coloring treatments.

In one embodiment of the present invention, the previous oxidative hair coloring treatment is an oxidative hair coloring treatment performed by a professional colorist in a hair salon. In another embodiment of the present invention, the previous oxidative hair coloring treatment is an at-home oxidative hair coloring treatment performed by using a retail hair coloring kit. In a further embodiment of the present invention, the retail hair coloring kit is one of the plurality of hair coloring products in kit form which are provided at the purchase location.

In an additional embodiment of the present invention, the purchase location is not a hair salon. In yet another embodiment of the present invention, the purchase location is selected from a retail store and an Internet website.

The hair color refresher product is provided in kit form and comprises a hair color refresher component and instruction for use. Additionally, the hair color refresher product may further comprise outer packaging, such as a cardboard or plastic box or other container, in which the other components are contained. The hair color refresher component is individually packaged, for example, in its own separate container. Contained within the hair color refresher component is a hair color refresher composition comprising at least one preformed or water soluble or water dispersible dye or mixtures thereof, together with a suitable solvent.

The hair color refresher product is provided in a plurality of general shades. According to the present invention, at least one shade among this plurality of general shades is suitable for refreshing the initial color of the hair of a particular prospective consumer.

In one embodiment of the present invention, one of the plurality of general shades of the hair color refresher product is selected from the group consisting of a general shade for pale to light blonde hair, a general shade for medium to dark blonde hair, a general shade for auburn hair, a general shade for dark blonde to light brown hair, a general shade for light to medium brown hair, a general shade for burgundy hair, and a general shade for dark brown hair.

1. Hair Color Refresher Composition

The hair color refresher composition comprises at least one preformed or water soluble or water dispersible dye or mixtures thereof. Dye compounds suitable for use in the hair color refresher composition of the present invention are known in the art and include acid dyes, basic dyes, disperse dyes, reactive dyes, and mixtures thereof. A representative but not exhaustive list can be found in (i) Colour Index 3rd Ed; Society of Dyers & Colourists: Bradford, West Yorkshire & the American Association of Textile Chemists & Colourists: Research Triangle Park 1971 and (ii) International Cosmetic Ingredient Dictionary and Handbook, 10th ed. (2004).

The hair color refresher compositions of the present invention comprise at least one preformed or water soluble or water dispersible dye or mixtures thereof in an amount ranging from about 0.01 wt. % to 10.0 wt. %. In one embodiment of the present invention, the hair color refresher compositions comprise from about 0.01 wt. % to 3.0 wt. % of at least one preformed or water soluble or water dispersible dye or mixtures thereof.

Non-limiting examples of suitable acid dyes include Acid Red 27 (C.I. 16185), Acid Red 51 (C.I. 45430), Acid Red 18 (C.I. 16255), Acid Red 92 (C.I. 45410), Acid Red 94 (C.I. 45440), Acid Red 52 (C.I. 45100), Acid Yellow 23 (C.I. 19140), Food Yellow 3 (C.I. 15985), Food Green 3 (C.I. 42053), Food Blue 2 (C.I. 42090), Acid Blue 74 (C.I. 73015), Pigment Red 57-1 (C.I. 15850), Acid Red 33 (C.I. 17200), Acid Red 87 (C.I. 45380), Acid Orange 7 (C.I. 15510), Acid Red 95 (C.I. 45425), Acid Yellow 73 (C.I. 45350), Acid Yellow 3 (C.I. 47005), Acid Green 25 (C.I. 61570), Solvent Green 7 (C.I. 59040), Solvent Violet 13, Acid Green 5 (C.I. 42095), Acid Blue 5 (C.I. 42052), Acid Blue 9 (C.I. 42090), Acid Orange 24 (C.I. 20170), Acid Violet 9 (C.I. 45190), Food Red 6 (C.I. 16155), Acid Red 26 (C.I. 16150), Food Red 1 (C.I. 14700), Acid Red 88 (C.I. 15620), Acid Orange 20 (C.I. 14600), Acid Yellow 40 (C.I. 18950), Acid Yellow 1 (C.I. 10316), Acid Yellow 36 (C.I. 13065), Acid Yellow 11 (C.I. 18820), Acid Green 1 (C.I. 10020), Acid Green 3 (C.I. 42085), Acid Violet 43 (C.I. 60730), Acid Black 1 (C.I. 20470), Acid Black 52 (C.I. 15711), Acid Blue 1 (C.I. 42045), Acid Blue 3 (C.I. 42051), Acid Blue 62 (C.I. 62045), Acid Brown 13 (C.I. 10410), Acid Green 50 (C.I. 44090), Acid Orange 3 (C.I. 10385), Acid Orange 6 (C.I. 14270), Acid Red 14 (C.I. 14720), Acid Red 35 (C.I. 18065), Acid Red 73 (C.I. 27290), Acid Red 184 (C.I. 15685), and Brilliant Black 1 (C.I. 28440).

Non-limiting examples of suitable basic dyes include Basic Blue 7 (C.I. 42595), Basic Blue 16 (C.I. 12210), Basic Blue 22 (C.I. 61512), Basic Blue 26 (C.I. 44045), Basic Blue 99 (C.I. 56059), Basic Blue 117, Basic Violet 10 (C.I. 45170), Basic Violet 14 (C.I. 42515), Basic Brown 16 (C.I. 12250), Basic Brown 17 (C.I. 12251), Basic Red 2 (C.I. 50240), Basic Red 12 (C.I. 48070), Basic Red 22 (C.I. 11055), Basic Red 51, Basic Red 76 (C.I. 12245), Basic Red 118 (C.I. 12251:1), Basic Orange 31, Basic Orange 69, Basic Yellow 28 (C.I. 48054), Basic Yellow 57 (C.I. 12719), Basic Yellow 87, and Basic Black 2 (C.I. 11825); basic dyes containing a quaternary nitrogen atom in the side chain of an aromatic ring skeleton, the dyes disclosed in, for example, Japanese Patent Publication (kokoku) No. 58-2204 and Japanese Patent Application Laid-Open (kokai) No. 9-118832; and basic dyes disclosed in, for example, Japanese Kohyo Patent Publication No. 10-502946 and Japanese Patent Application Laid-Open (kokai) Nos. 10-182379 and 11-349457.

Non-limiting examples of suitable preformed dyes other than acid dyes and basic dyes include 2-amino-3-nitrophenol, 2-amino-4-nitrophenol, 2-amino-5-nitrophenol, 4-amino-3-nitrophenol, 2-amino-6-chloro-4-nitrophenol, 4-hydroxypropylamino-3-nitrophenol, 3-nitro-p-hydroxyethylaminophenol, 2-nitro-p-phenylenediamine, 4-nitro-o-phenylenediamine, 4-nitro-m-phenylenediamine, 6-nitro-o-toluidine, 6-nitro-p-toluidine, hydroxyethyl-2-nitro-p-toluidine, N,N′-bis(2-hydroxyethyl)-2-nitro-p-phenylenediamine, 2-chloro-5-nitro-N-hydroxyethyl-p-phenylenediamine, 2-nitro-5-glycerylmethylaniline, 4-amino-2-nitrophenylamine-2-carboxylic acid, 2-chloro-6-ethylamino-4-nitrophenol, 3-methylamino-4-nitrophenoxyethanol, N-ethyl-3-nitro-PABA, picramic acid, 2-hydroxyethylpicramic acid, 4-nitrophenylaminoethylurea, Solvent Violet 13 (C.I. 60725), Solvent Yellow 44 (C.I. 56200), Disperse Red 17 (C.I. 11210), Disperse Violet 1 (C.I. 61100), Disperse Violet 4 (C.I. 61105), Disperse Black 9, Disperse Blue 377, Disperse Blue 23, Disperse Blue 3 (C.I. 61505), Disperse Blue 7 (C.I. 62500), HC Violet No. 1, HC Green No. 1, HC Blue No. 2, HC Blue No. 7, HC Blue No. 8, HC Blue No. 9, HC Blue No. 10, HC Blue No. 12, HC Blue No. 14, HC Orange No. 1, HC Orange No. 2, HC Orange No. 3, HC Red No. 1, HC Red No. 3, HC Red No. 7, HC Red No. 8, HC Red No. 9, HC Red No. 10, HC Red No. 11, HC Red No. 13, HC Red No. 14, HC Red No. 16, HC Violet No. 2, HC Yellow No. 2, HC Yellow No. 4, HC Yellow No. 5, HC Yellow No. 6, HC Yellow No. 7, HC Yellow No. 9, HC Yellow No. 10, HC Yellow No. 11, HC Yellow No. 12, HC Yellow No. 13, HC Yellow No. 14, and HC Yellow No. 15.

Among preferred preformed dyes for use herein are Acid Black 1, Acid Orange 3, Acid Red 18, Acid Red 92, Acid Violet 43, Acid Yellow 1, Basic Red 51, Basic Orange 31, Basic Yellow 57, Basic Yellow 87, Basic Blue 7, Basic Blue 99, Basic Brown 16, Basic Red 76, Basic Yellow 57, Basic Brown 17, Basic Brown 16, Basic Red 118, Basic Orange 69, Basic Violet 2, Curry Red, D&C Green 3, D&C Green 5, D&C Green 6, D&C Orange 4, D&C Red 33, D&C Yellow 10, Disperse Blue 3, Disperse Blue 3 (solvent form), Disperse Blue 377, Disperse Black 9, Disperse Red 17, Disperse Violet 1, FD&C Blue 1, FD&C Red 4, FD&C Red 30 lake, FD&C Yellow 5, FD&C Yellow 5 Zr Lake, FD&C Yellow 6, ex. FD&C Yellow 7, HC Blue 12, HC Blue 15, HC Orange 1, HC Red 1, HC Red 3, HC Red 10 & 11, HC Red 13, HC Blue 2, HC Yellow 2, HC Yellow 4, HC Yellow 13, HC Yellow 6, Pigment Red 57, Solvent Violet 13, 4-amino-3-nitrophenol, 3-nitro-p -hydroxyethylaminophenol, 4-nitrophenylaminoethylurea, 2-amino-6-chloro-4-nitrophenol, 6-amino-m-cresol, 2-nitro-p-phenylenediamine, picramic acid, 2-hydroxyethylpicramic acid, hydroxyethyl-2-nitro-p-toluidine, 2-chloro-6-ethylamino-4-nitrophenol, 2-amino-6-chloro-4-nitrophenol, 4-hydroxypropylamino-3-nitrophenol, 2,6-diamino-3-((pyridine-3-yl)azo)pyridine, tetrabromophenol Blue, and mixtures thereof.

In a preferred embodiment of the present invention, the hair color refresher composition, as it is applied to the hair, consists of no oxidizing agent, for example, hydrogen peroxide. Consequently, in a preferred embodiment of the present invention, the hair color refresher product consists of no individually packaged oxidizing agent composition as a component of the kit. Such hair color refresher products, which consist of no oxidizing agent, can be marketed to a prospective consumer as being gentle on the hair. This is advantageous for encouraging use of such a hair color refresher product in between periodic oxidative hair coloring treatments, which themselves can result in damage to the hair because of the use of oxidizing agents and optionally alkalizing agents. It is believed that a prospective consumer would be more likely to purchase and repeatedly use a hair color refresher product which the prospective consumer believes does not result in increased damage to the hair.

However, in an alternative embodiment of the present invention, the hair color refresher component comprises a hair color refresher composition comprising a combination of oxidative dyes and preformed dyes. Particularly preferable are combinations in the form of an aqueous or aqueous alcoholic solution comprising oxidative dye(s) and at least 3 preformed dyes whereby the weight ratio of oxidative dye to preformed dyes is 5:1 to 0.5:1. Such hair color refresher compositions, which comprise oxidative dyes and preformed dyes, may require the use of an oxidizing agent. Consequently, in this alternative embodiment of the present invention, the hair color refresher product may further comprise an oxidizing agent. Such an oxidizing agent may be contained in an individually packaged oxidizing agent composition as an additional component of the kit.

For use in such a combination of oxidative dyes and preformed dyes, non-limiting examples of suitable oxidative dyes include 2,5-diamino toluol, 2,4-diaminophenoxyethanol, resorcinol, 2-methylresorcinol, m-aminophenol, 4-amino-3-methylphenol, 4-amino-2-hydroxy-toluol, 6-amino-3-methyl phenol, 2-amino-4-hydroxyethylaminoanisol, 1-napthol, hydroxyethyl-3,4-methylenedioxyanilin, 2-5-diamino-phenolethanol, N,N-bis(2-hydroxyethyl)p-phenylenediamine, phenyl-methyl-pyrazolone, 1-hydroxyethyl-4,5-diaminopyrazol and 2-amino-chloro-4-vitro-phenol or the salts thereof. Suitable preformed dyes for use in such a combination with oxidative dyes include hydroxyethyl-2-nitro-ptoluidin, 2-hydroxyethyl-pikraminacid, 4-nitrophenyl-aminourea, basic violet 2, disperse violet 1, HC Blue No. 2, HC Blue No. 12, HC Red No. 13, HC Yellow No. 6, HC Red No. 3, 4-amino-3-nitrophenol, 4-[(hydroxyethyl)(-amino]-3-nitrophenol, HC Red No. 10, HC red No. 11, 2-chloro-6-ethylamino-4-nitrophenol, 2-amino-6-chloro-4-nitrophenol, HC Yellow No. 13, Basic Blue no. 99, Basic Brown No. 16, Basic Brown No. 17, Basic Yellow No 57, and 2,6,diamino-3[(pyridine-3-yl)azo]-pyridine, and the salts thereof.

Suitable solvents for use in the hair color refresher compositions of the present invention include, but are not limited to, water, butoxydiglycol, propylene glycol, alcohol (denat.), ethoxydiglycol, isopropylalcohol, hexylene glycol, benzyl alcohol and dipropylene glycol. The compositions of the present invention typically comprise from at least about 10%, preferably from about 20%, more preferably from about 30% and most preferably from about 50% by weight of solvent.

The hair color refresher compositions may further comprise surfactants. Anionic, cationic, nonionic, and zwitterionic surfactants, or mixtures thereof, can be used herein. When used in the hair color refresher compositions, anionic surfactants may be in an amount ranging from about 0.1 wt. % to about 30 wt. %, nonionic surfactants may be in an amount ranging from about 0.1 wt. % to about 20 wt. %, and zwitterionic surfactants may be in an amount ranging from about 0.1 wt. % to about 20 wt. %.

The hair color refresher compositions may optionally further comprise a functional silicone polymer. Suitable silicones for use in the hair color refresher composition are those silicones described in more detail below with respect to the Optional After-Treatment Conditioner.

The hair color refresher compositions according to the present invention may be provided in any suitable physical form, for example, as low to moderate to high viscosity liquids, lotions, milks, mousses, dispersions, sprays, gels, foams, aerosols, and creams. These forms may be produced by procedures well known to the skilled artisan.

2. Instructions for Use

The hair color refresher product in kit form also comprises instructions for use. The instructions for use, which may be written instructions optionally including images, direct a user of the hair color refresher product to apply the hair color refresher composition to the hair and to retain it on the hair for a period of time of about 15 minutes or less, and then to rinse the hair color refresher composition from the hair.

In one embodiment of the present invention, the instructions for use direct a user of the hair color refresher product to apply the hair color refresher composition to the hair and to retain it on the hair for a period of time ranging from about 3 minutes to about 9 minutes.

In a further embodiment of the present invention, the instructions for use direct a user of the hair color refresher product to apply the hair color refresher composition to the hair and to retain it on the for a period of time of about 5 minutes or less.

The instructions for use may further comprise additional information about proper use of the hair color refresher product.

3. Optional After-Treatment Conditioner

The hair color refresher product optionally may comprise an individually packaged after-treatment conditioner composition as a further component of the kit. Such a conditioner composition comprises one or more conditioning agents. Preferred conditioning agents include functionalized silicone polymers.

The conditioner composition comprises from about 0.1 wt. % to about 20 wt. %, preferably from about 0.2 wt. % to about 10 wt. %, more preferably from about 0.5 wt. % to about 7.5 wt. %, of the functionalized silicone.

The functionalized silicone polymers suitable for use herein have an interfacial tension of 1 to 12 mN/m, preferably from 1 to 10 mN/m, more preferably 1 to 8 mN/m, most preferably from 1 to 4 mN/m. The functionalized silicone polymers for use herein have a viscosity in the range 400 to 150,000 mPa·s. More advantageously, the viscosity is in the range 600 to 100,000 mPa·s. More advantageously still, the viscosity is in the range 4000 to 25,000 mPa·s.

The benefits associated with functionalized silicones having an interfacial tension and viscosity in the defined ranges apply regardless of chemistry, i.e., regardless of the functional groups concerned.

The conditioning compositions for use herein may comprise from about 0.1 to about 20 wt. %, preferably from about 0.25 to about 15 wt. %, more preferably from about 0.5 to about 10 wt. %, and more preferably still from about 0.5 wt. % to about 7.5 wt. % of the suitable functionalized silicone polymer.

Functionalized silicone polymers which may be incorporated into compositions according to the invention include organo-modified silicones of the pendant, graft or block type wherein these polar functional substituents for use in the present invention as described include, but are not limited to, polyoxyalkylene (polyether), primary and secondary amine, amide, quaternary ammonium, carboxyl, sulfonate, sulfate, carbohydrate, phosphate, and hydroxyl.

Suitable functionalized silicones of the present invention include, but are not limited to, organomodified silicones with amine functionality available commercially under the trade names such as ADM1100 and ADM1600 from Wacker Silicones, DC2-8211, DC8822, DC8822A, DC8803, DC2-8040, DC2-8813, DC2-8630 and DC8566 from Dow Corning Corporation, KF-862, KF-861, KF-862S, KF-8005, KF-8004, KF-867S, KF-873, and X-52-2328 from Shin-Etsu Corporation, and TSF 4702, TSF 4703, TSF 4704, TSF 4705, TSF 4707, TSF 4708, TSF 4709, F42-B3115, SF 1708, SF 1923, SF 1921, SF 1925, OF TP AC3309, OF 7747, OF-NH TP AI3631, OF-NH TP AI3683 from GE Bayer Silicones.

The functionalized silicones herein can be used together with a durability additive. The durability additive is capable of modifying the functionalized silicones to render them more durable on polar fibrous substrates, especially where the substrate is hair that has been previously damaged through chemical treatments, such as occurs during permanent dyeing, bleaching and permanent waving.

Preferably, the durability additive according to the invention comprises one or more organosiloxane resins. Without wishing to be bound by theory, organosiloxane resins are believed to create a 3-dimensional network within the functionalized silicone fluid giving rise to viscoelasticity thereby improving the adhesive properties of the fluid and hence the durability on a fibrous substrate. Preferably, the organosiloxane resin is insoluble in water.

Organosiloxane resins such as these are commercially available as SR1000 available from GE Bayer Silicones and Wacker 803 from Wacker Silicones. Advantageously, the organosiloxane resins according to the invention are solid at about 25° C. and have a molecular weight range of from 1,000 to 10,000 grams/mole.

The conditioning composition according to the present invention may include a cosmetically acceptable vehicle to act as a diluent, dispersant, or carrier for the silicone oil in the composition, so as to facilitate the distribution of the silicone oil when the composition is applied. The vehicle may be an aqueous emulsion, water, liquid or solid emollients, solvents, humectants, propellants, thickeners, and powders.

4. Plurality of Oxidative Hair Coloring Products

The hair color refresher product in kit form is provided at a purchase location at which a plurality of oxidative hair coloring products in kit form also are provided. The oxidative hair coloring products include any retail product or kit which can be used by a consumer to perform a permanent (i.e., Level 3) or a demi-permanent (i.e., Level 2) at-home hair coloring treatment.

Typically, such oxidative hair coloring products comprise (i) a first individually packaged component comprising at least one oxidizing agent, and (ii) a second individually packaged component comprising at least one oxidative dye or at least one alkalizing agent or mixtures thereof. These oxidative hair coloring products and their basic components are generally known.

In one embodiment of the method of the present invention, each of the plurality of oxidative hair coloring products is provided in a respective specific shade which imparts a respective specific color to the hair of a user of that respective specific shade of oxidative hair coloring product. Additionally, the initial color of the hair of said prospective consumer is at least similar to the respective specific color imparted by the respective specific shade of at least one of said plurality of hair coloring products.

In another embodiment of the present invention, the initial color of the hair of the prospective consumer is the respective specific color imparted by the respective specific shade of one of the plurality of hair coloring products.

In a further embodiment of the present invention, each of the plurality of general shades of the hair color refresher product is suitable for refreshing the respective specific color imparted by the respective specific shade of more than one of the plurality of oxidative hair coloring products.

In yet another embodiment of the present invention, the plurality of oxidative hair coloring products comprises at least two different brands of hair color products.

In a further embodiment of the present invention, the plurality of oxidative hair coloring products comprises a first oxidative hair coloring product made by a first manufacturer and a second oxidative hair coloring product made by a second manufacturer.

In an even further embodiment of the present invention, the hair color refresher product and at least one of said plurality of oxidative hair coloring products both are made by the same manufacturer.

In an additional embodiment of the present invention, the hair color refresher product and at least one of the plurality of oxidative hair coloring products both are marketed under the same brand.

In another embodiment of the present invention, a hair color refresher product may be provided in a kit which also comprises an oxidative hair coloring product.

B. Communicating to the Prospective Consumer

The methods of the present invention also comprise the step of communicating to the prospective consumer when and why the hair color refresher product is used. As the hair color refresher product is a new-to-retail-product, this step of the method of the present invention educates a prospective consumer about how the new hair color refresher product can address a currently unmet consumer need. This, in turn, encourages a prospective consumer to purchase and to try the new hair color refresher product.

According to the present invention, it is communicated to a prospective consumer that the hair color refresher product is used on the hair of the prospective consumer at a time after the prospective consumer's previous oxidative hair coloring treatment but before a subsequent oxidative hair coloring treatment. Stated differently, the hair color refresher product is intended for use in between a consumer's regular or periodic oxidative hair coloring treatments.

It also is communicated to a prospective consumer, according to the present invention, that the hair color refresher product is used on the hair of the prospective consumer to refresh the initial color of the hair of the prospective consumer. As used herein, the term “to refresh” means to consumer-observably increase or enhance the color or the shine of a consumer's hair, to remedy any consumer-observable fading of the initial color or the initial shine observed immediately after a previous hair coloring treatment. Use of the hair color refresher product is not intended to be a substitute for a subsequent oxidative hair coloring treatment. Instead, use of the hair color refresher product is intended to improve the consumer-observable color and/or shine of the hair until the consumer's next oxidative hair coloring treatment.

Advantageously, for a manufacturer of the hair color refresher product who also manufactures oxidative hair coloring products, the increase or enhancement of the color or the shine of the hair provided by the hair color refresher product lasts for preferably about 6 to 8 shampoos. This may mitigate any extent to which providing the hair color refresher product cannibalizes the same manufacture's sales of oxidative hair coloring products.

In one embodiment of the method of the present invention, the time after the previous oxidative hair coloring treatment but before a subsequent oxidative hair coloring treatment is communicated as being a time at which the prospective consumer observes fading of the initial color or the initial shine of the hair of the prospective consumer.

In another embodiment of the method of the present invention, the time after the previous oxidative hair coloring treatment but before a subsequent oxidative hair coloring treatment is communicated as being within a period of time ranging from about 1 day to about 60 days after the time of the previous oxidative hair coloring treatment.

In a further embodiment of the method of the present invention, the time after the previous oxidative hair coloring treatment but before a subsequent oxidative hair coloring treatment is communicated as being within a period of time ranging from about 15 days to about 30 days after the time of the previous oxidative hair coloring treatment.

The step of communicating to a prospective consumer may be carried out in any manner or by any means known to one of ordinary skill in the fields of advertising and marketing. For example, a suitable step of communicating may be effectuated with written statements or visual images, or combinations thereof, made on the hair color refresher product or on in-store promotional items and displays, as well as in magazines or newspapers and on television or the Internet, and the like. Similarly, oral statements by a promoter in-store or on the radio, television, the Internet, and the like, are suitable.

In one embodiment of the present invention, the step of communicating to a prospective consumer comprises advertising in a medium selected from television, radio, print, and the Internet.

In another embodiment of the present invention, wherein the hair color refresher product further comprises outer packaging, the step of communicating to a prospective consumer comprises providing written statements or visual images or combinations thereof on the outer packaging or on in-store promotional items.

The exact verbiage of the written statements, the exact portrayal of the visual images, etc., may be determined by one of ordinary skill in the fields of advertising and marketing.

A non-limiting example of a suitable written statement communicating to a prospective consumer why the new hair color refresher product is used is: “Love that just-colored shine, but it's too soon to re-color? The First-Ever Salon-Inspired Hair Color Refresher Product. Your simple, no fuss answer.”

A non-limiting example of a written statement communicating to a prospective consumer when and why the new hair color refresher product is used is: “When should I use the Hair Color Refresher Product? Use at the first sign of color fade. You'll get a beautiful boost of color and shine in-between your regular colorings.”

C. Providing Indicia to Facilitate Selection

In some embodiments, the methods of the present invention also comprise the step of providing one or more indicia in association with each of the plurality of general shades of the hair color refresher product. Doing so facilitates selection by the prospective consumer of one of the plurality of general shades of the hair color refresher product which is suitable for refreshing the initial color or the initial of the hair of the prospective consumer. By assisting a consumer in selecting a shade of the new hair color refresher product suitable to provide the results desired by the consumer, it is believed that a consumer is more confident about purchasing the new product for the first time. Moreover, it is also believed that the consumer is more likely to be satisfied with the performance of the new product, and, thus, is more likely to purchase the new product repeat times.

The step of providing one or more indicia in association with each of the plurality of general shades of the hair color refresher composition may be carried out in any manner or by any means known to one of ordinary skill in the fields of advertising and marketing. As used herein, the term “indicia” refers to any identifying mark which is useful to a prospective consumer in distinguishing one of the plurality of general shades of the hair color refresher product from among the others, and, further which signals to the consumer that at least one of the plurality of general shades of the hair color refresher product is suitable for refreshing the initial color or the initial shine of the hair of the consumer.

Suitable indicia include, for example, written statements, visual images, geometric shapes and patterns, specific colors, or combinations thereof, made on the hair color refresher product. Alternatively, such indicia made on in-store promotional items and displays, including on-shelf displays, which respectively are associated with one general shade of the hair color refresher product, also are suitable.

In one embodiment of the present invention, wherein the hair color refresher product further comprises outer packaging, the step of providing one or more indicia in association with each of the plurality of general shades of the hair color refresher product comprises providing written statements, visual images, or geometric shapes, or combinations thereof, on the outer packaging.

As with the step of communicating above, the exact verbiage of the written statements, the exact portrayal of the visual images, etc., may be determined by one of ordinary skill in the fields of advertising and marketing.

A non-limiting example of suitable indicia for the general shade for auburn hair is a colored circle, which contains within the circle the written statement “For Auburn”, and which is prominently placed on the front face of the outer packaging of the hair color refresher product. This example is equally applicable to each of the other general shades of the hair color refresher product by substituting the written statement within the circle with the appropriate general shade name or description.

### NON-LIMITING EXAMPLES

The following examples illustrate the hair color refresher compositions according to the present invention. It is understood that the examples and embodiments described herein are for illustrative purposes only and that various modifications or changes in light thereof will be suggested to one skilled in the art without departing from the scope of the present invention.

These exemplified embodiments of the hair color refresher composition of the present invention can refresh certain initial colors of hair along with initial shine, and can be merchandised as part of a hair color refresher product according to the methods of the present invention.

| Color Refresher Composition: Shampoo | | | |
|---|---|---|---|
| Ingredient | #1 | #2 | #3 |
| Aqua | qs | qs | qs |
| Sodium laureth sulphate | 10 | 10 | 10 |
| Peg-3 distearate | 2 | 2 | 2 |
| Cocoamidopropyl betaine | 4 | 4 | 4 |
| PEG-200 Hydrogenated glceryl | 1 | 1 | 1 |
| palmate | | | |
| Denat. alcohol1.90000 | 2 | 2 | 2 |
| Sodium chloride | 1 | 1 | 1 |
| Phenoxyethanol | 0.5 | 0.5 | 0.5 |
| Methyl paraben | 0.2 | 0.2 | 0.2 |
| Propylparaben | 0.1 | 0.1 | 0.1 |
| Perfume | 0.3 | 0.3 | 0.3 |
| HC Blue #2 | 0.2 | 0.2 | 0.3 |
| HC Red #10 | 0.09 | — | 0.2 |
| HC Red #11 | 0.03 | — | — |
| Hhydroxyethyl-2-Nitro-P-toluidine | 0.1 | — | — |

| Color Refresher Composition: Cream | Dark Brown | Light Brown |
|---|---|---|
| Ingredient | shade | shade |
| Aminomethyl Propanol | 1.5 | 2.5 |
| Diethylene Glycol Monoethyl Ether | 4 | 4 |
| PEG-50 Palm Amide | 2 | 2 |
| Lauryl Polyglucose* | 1 | 1 |
| Erythorbic Acid | 0.025 | 0.025 |
| HC Yellow No. 2 | 0.16 | 0.02 |
| Disperse Black 9 | 0.02 | 0.02 |
| HC Red No. 3 | 0.1 | 0.0113 |
| Disperse Violet 1 | 0.1 | — |
| HC Blue No. 2 | 0.9 | 0.1 |
| Disperse Blue 377 | 0.23 | 0.01 |
| HC Orange No. 1 | 0.0126 | 0.01 |
| Deionized Water | QS | QS |
| Citric Acid | 0.5 | 0.5 |
| Isopropyl Alcohol | 0.8 | — |
| Hydroxyethylcellulose* | 0.9 | — |
| Carbomer* | — | 0.55 |
| Trimethylsilylamodimethicone* | 2 | 2 |
| Fragrance 50854M | 1 | 1 |
| Glycerin | 0.2 | 0.2 |
| Hydrolyzed Whole Wheat Protein | 0.1 | 0.1 |

*Lauryl Polyglucose: trade name is Plantaren 1200N from Cognis
*Hydroxyethylcellulose: trade name is Natrosol from Hercules
*Carbomer: trade name is Carbopol Ultrez 10 Polymer from Noveon
*Trimethylsilylamodimethicone is conditioning additive Q2-8220 from Dow Corning

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of merchandising a hair color refresher product to a prospective consumer having oxidatively colored hair of an initial color imparted by a previous oxidative hair coloring treatment, said method comprising the steps of:
a) providing said hair color refresher product in kit form at a retail purchase location at which a plurality of oxidative hair coloring products in kit form also are provided, said hair color refresher product comprising:
i) an individually packaged hair color refresher component comprising a hair color refresher composition, said hair color refresher composition comprising at least one preformed or water soluble or water dispersible dye, and
ii) instructions for use which direct a user of said hair color refresher product to apply said hair color refresher composition to the hair of said user and to retain said hair color refresher composition on the hair of said user for a period of time of about 15 minutes or less, and then to rinse said hair color refresher composition from the hair of said user, and
wherein said hair color refresher product is provided in a plurality of general shades, at least one of said plurality of general shades being suitable for refreshing said initial color of the hair of said prospective consumer;
b) communicating to said prospective consumer that said hair color refresher product is used on the hair of said prospective consumer, at a time after said previous oxidative hair coloring treatment but before a subsequent oxidative hair coloring treatment, to refresh said initial color of the hair of said prospective consumer; and
c) providing one or more indicia in association with each of said plurality of general shades of said color refresher product to facilitate selection by said prospective consumer of one of said plurality of general shades of said hair color refresher product which is suitable for refreshing said initial color of the hair of said prospective consumer.

2. A method according to claim 1, wherein said instructions for use direct said user of said hair color refresher product to apply said hair color refresher composition to the hair of said user and to retain said hair color refresher composition on the hair of said user for a period of time ranging from about 3 minutes to about 9 minutes.

3. A method according to claim 1, wherein said instructions for use direct said user of said hair color refresher product to apply said hair color refresher composition to the hair of said user and to retain said hair color refresher composition on the hair of said user for a period of time of about 5 minutes or less.

4. A method according to claim 1, wherein said time after said previous oxidative hair coloring treatment but before a subsequent oxidative hair coloring treatment is communicated as being a time at which said prospective consumer observes fading of said initial color of the hair of said prospective consumer.

5. A method according to claim 1, wherein said time after said previous oxidative hair coloring treatment but before a subsequent oxidative hair coloring treatment is communicated as being within a period of time ranging from about 1 day to about 60 days after the time of said previous oxidative hair coloring treatment.

6. A method according to claim 5, wherein said time after said previous oxidative hair coloring treatment but before a subsequent oxidative hair coloring treatment is communicated as being within a period of time ranging from about 15 days to about 30 days after the time of said previous oxidative hair coloring treatment.

7. A method according to claim 1, wherein said hair color refresher product further comprises outer packaging, and wherein said step b) of communicating to said prospective consumer comprises providing written statements or visual images or combinations thereof on said outer packaging or on in-store promotional items.

8. A method according to claim 1, wherein said step b) of communicating to said prospective consumer comprises advertising in a medium selected from the group consisting of television, radio, print, and the Internet.

9. A method according to claim 1, wherein said hair color refresher product further comprises outer packaging, and wherein said step c) of providing one or more indicia in association with each of said plurality of general shades of said color refresher product comprises providing written statements, visual images, or geometric shapes, or combinations thereof, on said outer packaging.

10. A method according to claim 1, wherein one of said plurality of general shades of said hair color refresher product is selected from the group consisting of a general shade for pale to light blonde hair, a general shade for medium to dark blonde hair, a general shade for auburn hair, a general shade for dark blonde to light brown hair, a general shade for light to medium brown hair, a general shade for burgundy hair, and a general shade for dark brown hair.

11. A method according to claim 1, wherein each of said plurality of oxidative hair coloring products is provided in a respective specific shade which imparts a respective specific color to the hair of a user of that respective specific shade of oxidative hair coloring product, and wherein said initial color of the hair of said prospective consumer is at least similar to the respective specific color imparted by the respective specific shade of at least one of said plurality of hair coloring products.

12. A method according to claim 11, wherein said initial color of the hair of said prospective consumer is the respective specific color imparted by the respective specific shade of one of said plurality of oxidative hair coloring products.

13. A method according to claim 11, wherein each of said plurality of general shades of said hair color refresher product is suitable for refreshing the respective specific color imparted by the respective specific shade of more than one of said plurality of oxidative hair coloring products.

14. A method according to claim 1, wherein said plurality of oxidative hair coloring products comprises at least two different brands of hair color products.

15. A method according to claim 1, wherein said plurality of oxidative hair coloring products comprises a first oxidative hair coloring product made by a first manufacturer and a second oxidative hair coloring product made by a second manufacturer.

16. A method according to claim 1, wherein said hair color refresher product and at least one of said plurality of oxidative hair coloring products both are made by the same manufacturer.

17. A method according to claim 16, wherein said hair color refresher product and at least one of said plurality of oxidative hair coloring products both are marketed under the same brand.

18. A method according to claim 1, wherein said previous oxidative hair coloring treatment is an oxidative hair coloring treatment performed by a professional colorist in a hair salon.

19. A method according to claim 1, wherein said previous oxidative hair coloring treatment is an at-home oxidative hair coloring treatment performed by using a retail hair coloring kit.

20. A method according to claim 19, wherein said retail hair coloring kit is one of said plurality of oxidative hair coloring products in kit form which are provided at said purchase location.

21. A method according to claim 1, wherein said purchase location is not a hair salon.

22. A method according to claim 1, wherein said purchase location is selected from the group consisting of a retail store and an Internet website.

23. A method of merchandising a hair color refresher product to a prospective consumer having oxidatively colored hair of an initial color and an initial shine imparted by a previous oxidative hair coloring treatment, said method comprising the steps of:
a) providing said hair color refresher product in kit form at a retail purchase location at which a plurality of oxidative hair coloring products in kit form also are provided, said hair color refresher product comprising:
i) an individually packaged hair color refresher component comprising a hair color refresher composition, said hair color refresher composition comprising at least one preformed or water soluble or water dispersible dye, and
ii) instructions for use which direct a user of said hair color refresher product to apply said hair color refresher composition to the hair of said user and to retain said hair color refresher composition on the hair of said user for a period of time of about 15 minutes or less, and then to rinse said hair color refresher composition from the hair of said user, and
wherein said hair color refresher product is provided in a plurality of general shades, at least one of said plurality of general shades being suitable for refreshing said initial color and said initial shine of the hair of said prospective consumer;
b) communicating to said prospective consumer that said hair color refresher product is used on the hair of said prospective consumer, at a time after said previous oxidative hair coloring treatment but before a subsequent oxidative hair coloring treatment, to refresh said initial color and said initial shine of the hair of said prospective consumer; and
c) providing one or more indicia in association with each of said plurality of general shades of said color refresher product to facilitate selection by said prospective consumer of one of said plurality of general shades of said hair color refresher product which is suitable for refreshing said initial color of the hair of said prospective consumer.

24. A method of merchandising a hair color refresher product to a prospective consumer having oxidatively colored hair of an initial color imparted by a previous oxidative hair coloring treatment, said method comprising the steps of:
a) providing said hair color refresher product in kit form at a retail purchase location at which a plurality of oxidative hair coloring products in kit form also are provided, said hair color refresher product comprising:
i) an individually packaged hair color refresher component comprising a hair color refresher composition, said hair color refresher composition comprising at least one preformed or water soluble or water dispersible dye, and
ii) instructions for use which direct a user of said hair color refresher product to apply said hair color refresher composition to the hair of said user and to retain said hair color refresher composition on the hair of said user for a period of time of about 15 minutes or less, and then to rinse said hair color refresher composition from the hair of said user; and
b) communicating to said prospective consumer that said hair color refresher product is used on the hair of said prospective consumer, at a time after said previous oxidative hair coloring treatment but before a subsequent oxidative hair coloring treatment, to refresh said initial color of the hair of said prospective consumer.

25. A method according to claim 24, wherein said time after said previous oxidative hair coloring treatment but before a subsequent oxidative hair coloring treatment is communicated as being a time at which said prospective consumer observes fading of said initial color of the hair of said prospective consumer.
